(19) United States
(12) Patent Application Publication
(10) Pub. No.: US 2009/0137831 A1
(43) Pub. Date: May 28, 2009

(54) Fluorinated Alkanesulfonic Acid Esters and Amides and Processes for Making and Using the Same

(75) Inventor: Vsevolod Rostovtsev, Swarthmore, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPANY, Wilmington, DE (US)

(21) Appl. No.: 12/274,655

(22) Filed: Nov. 20, 2008

Related U.S. Application Data
(60) Provisional application No. 60/990,745, filed on Nov. 28, 2007.

(51) Int. Cl. C07C 303/26; C07C 303/36

(52) U.S. Cl. 558/48; 558/54; 564/96; 564/97

(57) ABSTRACT

The invention provides novel aryl and aliphatic esters of fluorinated alkanesulfonic acids. The invention also provides novel fluorinated alkanesulfonamides and a process to make the same. The invention also provides a process for the arylation of an amine by contacting the amine with an aryl ester of a fluorinated alkanesulfonic acid.

### BACKGROUND OF THE INVENTION

The present invention relates to fluorinated alkanesulfonic acid esters and amides and processes for making and using such esters and amides.

Because the perfluoroalkanesulfonate anion is an effective leaving group in substitution reactions, perfluoroalkanesulfonic acid esters are used in the alkylation of amines and heterocycles among other applications (see Stang, Hanack, and Subramanian, Synthesis, 1982, issue 2, pp. 85-126). The esters can be made in several ways, but it is convenient to make them by reaction of alcohols with perfluoroalkanesulfonic acid anhydrides.

Common perfluoroalkanesulfonic acids are trifluoromethanesulfonic acid (triflic acid) and nonafluoro-n-butanesulfonic acid (nonaflic acid). The reactions of aliphatic alcohols with the anhydrides of triflic acid and nonaflic acid form the corresponding alkyl esters (i.e., alkyl triflates and alkyl nonaflates, respectively). Lately however, there is growing interest in finding alkanesulfonic acids that are not perfluorinated, but whose esters are still effective alkylating agents. Esters of partially fluorinated alkanesulfonic acids satisfy this criterion.

1-Hydrotetrafluoroethanesulfonic acid anhydride (I) reacts with

(CF₃CHFSO₂)₂O (I)

methanol and ethanol to form the corresponding esters, CF₃CHFSO₃R (R═CH₃ and C₂H₅), but with higher alcohols, elimination to form olefins and 1-hydrotetrafluoroethanesulfonic acid is the predominant reaction pathway. (see L. I. Ragulin, P. P. Ropalo, G. A. Sokol'skii, and I. L. Knunyants, Izvestiya Akademii Nauk SSR, Seriya Khimicheskaya, No. 7, pp. 1560-1564, July 1968).

Furthermore, reaction of 1-hydrotetrafluoroethanesulfonic acid anhydride with an amine gives not only the expected amide (II)

CF₃CHFSO₂NR₂ (II)

but also the diadduct (III) (β-aminotetrafluoroethanesulfonamide), often in higher yield than the amide (II):

R₂NCF₂CF₂SO₂NR₂ (III)

(see I. L. Knunyants and G. A. Sokolski, Angew. Chem. Internat. Edit., vol. 11, no. 7, p. 589 (1972)).

Aryl esters of perfluorinated alkanesulfonic acids are important intermediates for aromatic ring substitution. For example, the 4-t-butylphenyl ester of nonafluorobutanesulfonic acid (IV) reacts with aniline in the presence of a palladium catalyst and a base to give arylated amine (V) as disclosed by Tundel, et al. in Journal of Organic Chemistry, Vol. 71, pages 430-433 (2006).

4-t-butyl-C₆H₄—OSO₂C₄F₉ (IV)

4-t-butyl-C₆H₄—NH—C₆H₅ (V)

There is a need for partially fluorinated alkanesulfonic acid esters and amides that do not have the problems in preparation associated with using known 1-hydrofluoroalkanesulfonic acid anhydrides.

### SUMMARY OF THE INVENTION

The present invention provides novel alkyl esters of fluorinated alkanesulfonic acids having the formula R^{f}CHFCF₂SO₂OR⁴ where R^{f} is selected from the group consisting of Cl, F, a C₁ to C₄ perfluoroalkyl group, or a C₁ to C₄ perfluoroalkoxy group, and where R⁴ is a C₁ to C₁₂ linear, branched, or cyclic alkyl group or an C₇ to C₂₀ aryl-substituted alkyl group, and where the aryl group may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, and where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H (hydrogen) and R². The invention is based on the discovery that fluorinated 2-hydro alkanesulfonic acid anhydrides of the formula (R^{f}CHFCF₂SO₂O)O where R^{f} is as defined above, can be reacted with aliphatic alcohols, R⁴OH where R⁴ is as defined above, to form alkyl esters without significant olefin formation.

The present invention further provides novel aryl esters of fluorinated alkanesulfonic acids having the formula R^{f}CHFCF₂SO₂OR¹ where R^{f} is selected from the group consisting of Cl, F, a C₁ to C₄ perfluoroalkyl group, or a C₁ to C₄ perfluoroalkoxy group, and where R¹ is a C₆ to C₁₄ aryl group, and where the aryl group may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H (hydrogen) and R².

The present invention further provides a process for preparing alkyl esters of the formula R^{f}CHFCF₂SO₂OR⁴ where R^{f} is selected from the group consisting of Cl, F, a C₁ to C₄ perfluoroalkyl group, or a C₁ to C₄ perfluoroalkoxy group, and where R⁴ is a C₁ to C₁₂ linear, branched, or cyclic alkyl group or an C₇ to C₂₀ aryl-substituted alkyl group, where the aryl group may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H and R², comprising contacting alcohol of the formula HOR⁴ with fluorinated alkanesulfonic acid anhydride of the formula (R^{f}CHFCF₂SO₂)₂O.

The present invention further provides a process for preparing aryl esters of the formula R^{f}CHFCF₂SO₂OR¹ where R^{f} is selected from the group consisting of Cl, F, a C₁ to C₄ perfluoroalkyl group, or a C₁ to C₄ perfluoroalkoxy group, and where R¹ is a C₆ to C₁₄ aryl group, and where the aryl group may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H (hydrogen) and R², comprising contacting aromatic alcohol of the formula HOR¹ with fluorinated alkanesulfonic acid anhydride of the formula (R^{f}CHFCF₂SO₂)₂O.

The present invention further provides novel fluorinated alkanesulfonamides having the formula R^{f}CHFCF₂SO₂NR⁵R⁶ where R^{f} is selected from the group consisting of Cl, F, a C₁ to C₄ perfluoroalkyl group, or a C₁ to C₄ perfluoroalkoxy group, and where R⁵ is a C₁ to C₁₂ linear, branched, or cyclic alkyl group, or a C₇ to C₂₀ aryl-substituted alkyl group, or a C₆ to C₁₄ aryl group, the aryl groups may be further substituted with one or more substituents selected from the group consisting of R², F, Cl, Br, I, NO₂, CN, OR, C(O)R², CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H (hydrogen), and where R⁶ is independently selected from the group consisting of H (hydrogen) and R⁵, and where R⁵ and R⁶ together may form a cyclic structure (e.g. morpholine or carbazole).

The present invention further provides a process for making fluorinated alkanesulfonamides of the formula R^{f}CHFCF₂SO₂NR⁵R⁶ by contacting an amine of the formula NHR⁵R⁶ with a fluorinated alkanesulfonic acid anhydride of the formula (R^{f}CHFCF₂SO₂)₂O, where R⁵, R⁶, and R^{f} are as defined above.

The present invention also provides a process for the arylation of an amine of the formula NHR⁵R⁶ comprising contacting the amine with an aryl ester of a fluorinated alkanesulfonic acid of the formula R^{f}CHFCF₂SO₂OR¹ in the presence of a palladium catalyst to form an arylated amine of the formula NR¹R⁵R⁶, where R¹, R⁵, R⁶, and R^{f} are as defined above.

The present invention also provides a process for the alkylation of an amine of the formula NHR⁵R⁶ comprising contacting the amine with an alkyl ester of a fluorinated alkanesulfonic acid of the formula R^{f}CHFCF₂SO₂OR⁴ to form an alkylated amine of the formula NR⁴R⁵R⁶, where R⁴, R⁵, R⁶, and R^{f} are as defined above.

### DETAILED DESCRIPTION OF THE INVENTION

The present invention relates to alkyl ester, aryl ester, and amide derivatives of fluorinated alkanesulfonic acids and related processes. These derivatives may be prepared from the corresponding fluorinated alkanesulfonic acid anhydrides. The fluorinated alkanesulfonic acid anhydrides useful for preparing the derivatives have the general formula (R^{f}CHFCF₂SO₂)₂O where R^{f} is selected from the group consisting of Cl, F, a C₁ to C₄ perfluoroalkyl group, or a C₁ to C₄ perfluoroalkoxy group. The fluorinated alkanesulfonic acid anhydrides may be prepared by contacting the corresponding fluorinated alkanesulfonic acids, R^{f}CHFCF₂SO₂OH, with phosphorus pentoxide (P₂O₅) in the presence of an inert oil.

Examples of fluorinated alkanesulfonic acid anhydrides that may be used in the process of this invention include (CHF₂CF₂SO₂)₂O, (CHClFCF₂SO₂)₂O, (CF₃CHFCF₂SO₂)₂0, (CF₃OCHFCF₂SO₂)₂O, (C₂F₅OCH FCF₂SO₂)₂O, and (C₃F₇OCH FCF₂SO₂)₂O.

The fluorinated alkanesulfonic acids used as starting materials for preparation of the fluorinated alkanesulfonic acid anhydrides of this invention may be prepared by methods known in the art. For example, the starting material for 2-hydrotetrafluoroethanesulfonic acid anhydride ((CHF₂CF₂SO₂)₂O), 2-hydrotetrafluoroethanesulfonic acid (CHF₂CF₂SO₂OH, also referred to herein as TFESA), may be prepared according to the process disclosed in U.S. Patent Application Publication No. 2006/0276671. In this process tetrafluoroethylene (TFE) is reacted with an aqueous solution of potassium sulfite. The reaction product, potassium 2-hydrotetrafluoroethanesulfonate, is then collected, dried, treated with oleum, and the TFESA product is recovered by distillation. Similarly, fluorinated alkanesulfonic acids of the formula R^{f}CHFCF₂SO₂OH where R^{f} is Cl, CF₃, OCF₃, OC₂F₅, and OCF₂C₂F₅, that is CHClFCF₂SO₂OH, CF₃CHFCF₂SO₂OH, CF₃OCHFCF₂SO₂OH, (C₂F₅OCHFCF₂SO₂)₂O, and (C₂F₅CF₂OCHFCF₂SO₂)₂O, may be prepared by the reaction of sodium sulfite or potassium sulfite with chlorotrifluoroethylene, hexafluoropropylene, perfluoro(methyl vinyl ether), perfluoro(ethyl vinyl ether), and perfluoro(n-propyl vinyl ether), respectively, followed by acidification.

The fluorinated alkanesulfonic acid anhydrides used as starting materials for preparation of the esters and amides of the present invention are prepared by contacting fluorinated alkanesulfonic acids with phosphorus pentoxide (P₂O₅), the contacting being advantageously carried out in the presence of an inert oil. By inert oil is meant a fluid that is unreactive with P₂O₅, fluorinated alkanesulfonic acid, and fluorinated alkanesulfonic acid anhydride. Additional characteristics are described below. The dispersions can be prepared by methods known to those skilled in the art. One particularly useful method of preparing dispersions is to add solid particulates to the inert oil mixed with a high shear mixer such as those commercially available from Hockmeyer Equipment Corporation, Harrison, N.J. Such dispersers are available in a variety of blade configurations such as saw tooth, ring blade and vane blade configurations. This operation is preferentially done prior to addition of the fluorinated alkanesulfonic acid to insure a good dispersion of the P₂O₅ reagent. These dispersions can be further mixed by applying higher energy dispersion methods such as sonication, homogenization or microfluidization. Contacting may be carried out by adding the fluorinated alkanesulfonic acid to a vigorously stirred mixture of P₂O₅ and the inert oil. Contact times sufficient for forming the fluorinated alkanesulfonic acid anhydride are typically from about 5 minutes to about 12 hours, preferably about 30 minutes to about six hours. Suitable temperatures for contacting the fluorinated alkanesulfonic acid and P₂O₅ in the inert oil are from about 10° C. to about 100° C., preferably from about 20° C. to about 80° C. The contacting may take place at atmospheric or subatmospheric pressure. If the contacting is performed at subatmospheric pressure, the pressure should be sufficient to maintain at least a portion of the fluorinated alkanesulfonic acid in the liquid phase. The molar ratio of P₂O₅ to fluorinated alkanesulfonic acid is typically from about 1:1 to about 5:1, preferably from about 2.5:1 to about 4:1. Large excesses of P₂O₅ are not beneficial and substoichiometric amounts (i.e., molar ratios of P₂O₅ to fluorinated alkanesulfonic acid of less than 1:1, e.g., 0.8:1) will result in incomplete conversion of the fluorinated alkanesulfonic acid.

The contacting of fluorinated alkanesulfonic acid with P₂O₅ and the inert oil is carried out in a well-agitated reaction vessel suitable for containing highly acidic materials under reduced pressure. The vessels may be fabricated from glass, including glass-lined metal reactors, ceramic, or acid-resistant alloys such as Hastelloy™ C. The reaction vessels and supporting equipment such as mixers and product receivers should be substantially moisture-free.

The ratio of the volume of the inert oil dispersant relative to the weight of P₂O₅ reactant is typically from about 0.75:1 to about 5:1, preferably from about 1:1 to about 2.5:1.

Inert oils suitable for the process of the present invention are those which are stable to highly acidic materials such as fluorinated alkanesulfonic acids and phosphoric acid. Such acid-resistant oils include hydrocarbon oils such as mineral oils (e.g., TW fluids (Inland Vacuum Industries, Churchville, N.Y.)), perfluorinated oils such as perfluoro(polyethers) (e.g., low viscosity Krytox® oils (DuPont, Wilmington, Del.) or Fomblin™ oils (Solvay Solexis, Thorofare, N.J.)), or chlorofluorocarbon oils (e.g., Halovac® 100 or 125 (Inland Vacuum Industries). Other oils suitable for this invention are mixtures of diphenyl oxide and biphenyl, di- and trialkyl ethers, and alkylated aromatics commercially available as Dowtherm® (Dow Chemical, Midland, Mich.). Other oils suitable for this invention are polysiloxanes, especially polydimethyl siloxanes of molecular weights greater than 2,000 (fluids with viscosities greater than 50 cSt (50 mm²/s)). These materials are available from Dow Corning (Midland, Mich.) as DC-200 fluids. Another class of polysiloxanes useful for this invention are mixed methyl- and diphenyl fluids sold commercially as DC-550 and DC-710 by Dow Corning.

Inert oils suitable for the process of the present invention are further characterized by low vapor pressures in order to permit easy separation of the fluorinated alkanesulfonic acid anhydride product from the oil. Preferred oils have vapor pressures at 25° C. of no greater than about 1×10⁻³ torr (133 mPa), more preferably no greater than about 1×10⁻⁴ torr (13 mPa), and most preferably no greater than about 1×10⁻⁵ torr (1.3 mPa). Typically, these oils have boiling points greater than about 260° C., and preferably greater than 300° C. at atmospheric pressure.

The preferred oils for the preparation of fluorinated alkanesulfonic acid anhydrides of the present invention are chlorofluorocarbon oils. The chlorofluorocarbon oil preferably contains at least about 20 wt % chlorine, more preferably at least about 30 wt % chlorine, and most preferably at least about 40 wt % chlorine, and preferably no more than about 80 wt % chlorine, more preferably no more than about 70 wt % chlorine, and most preferably no more than about 60 wt % chlorine. The non-chlorine monovalent substituents are fluorine and hydrogen, preferably at least as many fluorine atoms as hydrogen atoms, more preferably, at least twice as many fluorine atoms as hydrogen atoms, still more preferably at least four times as many fluorine atoms as hydrogen atoms, and most preferably, only fluorine atoms, with no hydrogen in the chlorofluorocarbon oil.

Typically, the fluorinated alkanesulfonic acid anhydride is recovered by distillation. After contacting the reactants for a period of time sufficient to react at least a substantial portion of the fluorinated alkanesulfonic acid (e.g., greater than 90% of the fluorinated alkanesulfonic acid), the pressure in the reactor is adjusted to a value suitable for distillation of the fluorinated alkanesulfonic acid anhydride product. Suitable pressures for recovering the anhydride product are from about 100 millitorr (1.33 Pa) to about 50 torr (6.665 kPa), preferably from about 1 torr (13.3 Pa) to about 10 torr (133 Pa) at temperatures of from about 80° C. to about 120° C. Times of from about 0.5 to about 20 hours are sufficient for the reaction forming the anhydride to take place. Preferred times are from about 0.5 to about 5 hours. The temperature and pressure suitable for recovery of a particular fluorinated alkanesulfonic acid anhydride will depend on the vapor pressure of each product. However, distillation temperatures higher than about 120° C. are to be avoided because decomposition of the fluorinated alkanesulfonic acid anhydride typically becomes significant above about 120° C.

The fluorinated alkanesulfonic acid anhydride recovered in this manner is typically sufficiently pure for use as a starting material in other reactions such as the preparation of esters and amides. However, the recovered anhydride may be re-distilled if desired.

After the distillation of the anhydride product, the inert oil may be recovered from the reaction mixture for re-use in subsequent preparations. In one embodiment, the mixture remaining in the reactor after step (b) is treated with water, preferably with cooling. The inert oil and resulting aqueous phosphoric acid solution form separate liquid phases. The density of the inert oil will determine whether it is present as the upper or lower phase in the water-treated mixture. The inert oil is then separated (e.g., by decantation). The recovered oil may be washed with additional portions of water and, optionally, with an aqueous solution of a base such as potassium phosphate, sodium hydroxide, or the like, and then dried by heating at temperatures of from about 50° C. to about 120° C. preferably under vacuum at pressures of from about 1 torr (13.3 Pa) to about 100 torr (1.33 kPa).

The present invention provides novel aryl esters of fluorinated alkanesulfonic acids having the formula R^{f}CHFCF₂SO₂OR¹ where R^{f} is selected from the group consisting of Cl, F, a C₁ to C₄ perfluoroalkyl group, or a C₁ to C₄ perfluoroalkoxy group, and where R¹ is a C₆ to C₁₄ aryl group, and where the aryl group may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H (hydrogen) and R².

The novel aryl esters of the present invention may be prepared by reacting an aromatic alcohol (i.e., a derivative of phenol) of the formula HOR¹ with a fluorinated alkanesulfonic acid anhydride of the formula (R^{f}CHFCF₂SO₂)₂O, preferably in the presence of a base, where R^{f} and R¹ are as defined above.

The molar ratio of the fluorinated alkanesulfonic acid anhydride to base is typically from about 0.8:1 to about 1:2, preferably about 1:1. Typically, the fluorinated alkanesulfonic acid anhydride is dissolved or suspended in a non-reactive solvent such as dichloromethane, chloroform, chlorobenzene, toluene, benzotrifluoride, or the like, at a temperature of from about −10° C. to about −40° C., preferably from about −20° C. to about −30° C. The mixture of the anhydride in the solvent is then treated with a base. Suitable bases for the process include nitrogen-containing Lewis bases such as nitrogen heterocycles (e.g. pyridine or 1,8-diazabicyclo[5.4.0]undec-7-ene (DBU)) or a tertiary amine (e.g., triethylamine, 1,4-diazabicyclo[2.2.2]octane (DABCO), or 4-dimethylaminopyridine (DMAP)). The aromatic alcohol may be added as a solution (typically in the same solvent used for the fluorinated alkanesulfonic acid anhydride and base) to the mixture of the fluorinated alkanesulfonic acid anhydride and base with stirring. The rate of aromatic alcohol addition is such that the reaction temperature is maintained at from about −30° C. to about −10° C. When addition is complete, stirring is continued for an additional 30 minutes to 6 hours at about −10° C. to about 0° C.

The molar ratio of aromatic alcohol to the fluorinated alkanesulfonic acid anhydride is typically from about 1:1 to about 1:2, preferably about 1:1.5.

The product aryl ester may be isolated from the reaction mixture using techniques well-known in the art. These techniques may include an aqueous work-up and extraction or, in the case of hydrolytically sensitive materials, distillation.

Examples of aryl esters in accordance with this invention include CHF₂CF₂SO₂OC₆H₄-4-tert-C₄H₉, CHClFCF₂SO₂OC₆H₄-4-OCH₃, CF₃CH FCF₂SO₂OC₆H₄CF₃CH FCF₂SO₂OC₆H₄-3-CF₃, CF₃OCHFCF₂SO₂O-2-C₁₀H₇, C₂F₅OCHFCF₂SO₂OC₆H₄-4-CN, and C₂F₅CF₂OCHFCF₂SO₂OC₆H₅.

The present invention provides novel alkyl esters of fluorinated alkanesulfonic acids having the formula R^{f}CHFCF₂SO₂OR⁴ where R^{f} is selected from the group consisting of Cl, F, a C₁ to C₄ perfluoroalkyl group, or a C₁ to C₄ perfluoroalkoxy group, and where R⁴ is a C₁ to C₁₂ linear, branched, or cyclic alkyl group or an C₇ to C₂₀ aryl-substituted alkyl group, where the aryl group may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H and R².

The novel alkyl esters of the present invention may be prepared by adding an aliphatic alcohol of the formula HOR⁴ to a fluorinated alkanesulfonic acid anhydride of the formula (R^{f}CHFCF₂SO₂)₂O in the presence of a base where R^{f} and R⁴ are as defined above. The alkyl esters may be prepared by the procedures suitable for the aryl esters discussed above. In addition, it has been discovered that fluorinated alkanesulfonic acid anhydrides can be reacted with aliphatic alcohols higher than methanol and ethanol (e.g., n-butanol) to form alkyl esters of fluorinated alkanesulfonic acid without significant olefin formation.

Examples of alkyl esters in accordance with this invention include CHF₂CF₂SO₂O-n-C₄H₉, CHClFCF₂SO₂OCH₂CH₂Cl, CF₃CHFCF₂SO₂OCH₂CH₂C₆H₅, CF₃OCHFCF₂SO₂O-2-C₃H₇, C₂F₅OCHFCF₂SO₂OCH₃, and C₂F₅CF₂OCHFCF₂SO₂OCH₂CH₂C₆F₁₃.

The present invention further provides novel fluorinated alkanesulfonamides having the formula R^{f}CHFCF₂SO₂NR⁵R⁶ where R^{f} is selected from the group consisting of Cl, F, a C₁ to C₄ perfluoroalkyl group, or a C₁ to C₄ perfluoroalkoxy group, and where R⁵ is a C₁ to C₁₂ linear, branched, or cyclic alkyl group, or a C₇ to C₂₀ aryl-substituted alkyl group, or a C₆ to C₁₄ aryl group and where the aryl groups may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² and R³ are as defined above and where R⁶ is independently selected from the group consisting of H (hydrogen) and R⁵, and where R⁵ and R⁶ together may form a cyclic structure (e.g. morpholine or carbazole).

The fluorinated alkanesulfonamides may be prepared by reacting a fluorinated alkanesulfonic acid anhydride with an amine. The present invention thus provides a process for preparing fluorinated alkanesulfonamides of the formula R^{f}CHFCF₂SO₂NR⁵R⁶ by contacting a fluorinated alkanesulfonic acid anhydride of the formula (R^{f}CHFCF₂SO₂)₂O with an amine of the formula NHR⁵R⁶ where R^{f}, R⁵, and R⁶ are as defined above. The process can be carried out with little or no formation of fluorinated β-aminoalkanesulfonamides (VI).

R⁵R⁶NCF(R^{f})F₂SO₂NR⁵R⁶ (VI)

The fluorinated alkanesulfonamides may be prepared by the procedures suitable for the aryl esters discussed above except that the amine itself may serve as the base. Thus, if the amine is used as the base, the molar ratio of the amine to the fluorinated alkanesulfonic acid anhydride is typically from about 1.5:1 to about 3:1, preferably about 2:1.

Examples of alkanesulfonamides in accordance with this invention include CHF₂CF₂SO₂N(CH₃)₂, CHClFCF₂SO₂NH(tert-C₄H₉), CF₃CHFCF₂SO₂NHC₆H₅, CF₃OCHFCF₂SO₂N(C₂H₅)₂, C₂F₅OCHFCF₂SO₂NH(C₆H₄-3-CF₃), and C₂F₅CF₂OCHFCF₂SO₂NHC₆H₄-4-C₆H₅.

The novel aryl esters of the fluorinated alkanesulfonic acids of the present invention, R^{f}CHFCF₂SO₂OR¹, may be used as reagents for the arylation of amines of the formula NHR⁵R⁶ where R^{f}, R¹, R⁵ and R⁶ are as defined above. Thus, the present invention also provides a process for the arylation of an amine, NHR⁵R⁶, comprising contacting the amine with an aryl ester of a fluorinated alkanesulfonic acid of the formula R^{f}CHFCF₂SO₂OR¹ to form an N-aryl substituted amine of the formula NR¹R⁵R⁶. The contacting may be carried out using a catalyst and conditions as described for perfluoroalkanesulfonic acid esters by Anderson et al., in Journal of Organic Chemistry, Volume 68 pages 9563-9574 (2003) and Tundel, et al. in the Journal of Organic Chemistry, Volume 71, pages 430-433 (2006). As illustrated in Example 7, such an arylation takes place in the presence of a suitable catalyst prepared by mixing a palladium compound (e.g., Pd₂(dibenzylideneacetone)₃) with a ligand (e.g., tri-t-butyl phosphine).

It is noted that the thermal properties of aryl esters of 2-hydrotetrafluoroethanesulfonic acid may allow the use of its derivatives at temperatures higher than those used for reactions of aryl esters of trifluoromethanesulfonic and nonafluorobutanesulfonic acids. For example, 4-nitrophenyl trifluoromethanesulfonate loses 10% of its weight at 105° C., 4-nitrophenyl nonafluorobutanesulfonate loses 10% at 125° C., but 4-nitrophenyl 2-hydrotetrafluoroethanesulfonate only reaches the same weight loss at 150° C. A similar trend is seen for 50% weight loss. Unexpectedly, the melting point of 4-nitrophenyl 2-hydrotetrafluoroethanesulfonate is lowest in this series (24° C.).

The novel alkyl esters of the fluorinated alkanesulfonic acids of the present invention, R^{f}CHFCF₂SO₂OR⁴, may be used as reagents for the alkylation of amines of the formula NHR⁵R⁶ where R^{f}, R⁴, R⁵, and R⁶ are as defined above. Thus, the present invention also provides a process for the alkylation of an amine, NHR⁵R⁶, comprising contacting the amine with an alkyl ester of a fluorinated alkanesulfonic acid, R^{f}CHFCF₂SO₂OR⁴ to form an N-alkyl substituted amine of the formula NR⁴R⁵R⁶. The contacting may be carried out using a catalyst and conditions as described for perfluoroalkanesulfonic acid esters by Stang, et al. in Synthesis 1982, issue 2, pages 85-126.

### EXAMPLES

### Example 1

### Preparation of 2-Hydrotetrafluoroethanesulfonic acid anhydride

An oven-dried 500 mL round-bottom flask is charged with 40 g of sand, 82.0 g (57.7 mmol) of phosphorus pentoxide and a magnetic stir bar. The flask is swirled by hand, becoming warm to the touch. A short-path distillation column is attached and the reaction flask is evacuated and filled with nitrogen atmosphere, twice. 2-Hydrotetrafluoroethanesulfonic acid (41.09 g, 23.0 mmol) is added and the flask is evacuated and filled with nitrogen once more. The reaction mixture is warmed in a 65° C. oil bath and begins to turn dark brown. Reaction mixture is kept at 65° C. for three hours, followed by 16 hours at room temperature. Distillation is carried out at 75° C. under vacuum (60 mtorr, 7 Pa) giving 20.44 g of 2-hydrotetrafluoroethanesulfonic acid anhydride (44% yield), as a clear colorless liquid.

NMR Analysis: ¹H NMR (CD₂Cl₂) 6.31 (2H, tt, ²J_{HF}=51.6 Hz, ³J_{HF}=4.3 Hz). ¹⁹F NMR (CD₂Cl₂) −113.4 (4F, m); −135.5 (4F, dt, ²J_{HF}=51.8 Hz, ³J_{FF=}6.2 Hz).

### Example 2

### Preparation of 2-Hydrotetrafluoroethanesulfonic Acid Anhydride in Chlorofluorocarbon Oil

An oven-dried glassware 2 L, 3 neck round-bottom flask is equipped with a mechanical stirrer having a ⅜ inch (9.5 mm) thick Teflon® paddle attached to the shaft, a simple distillation apparatus attached to a recirculating chiller set to −5° C., and a nitrogen/vacuum inlet. The atmosphere in the flask is replaced with nitrogen. The flask is charged with 800 mL of Halovac® 100 chlorofluorocarbon oil and internal pressure is lowered to 2 torr (267 Pa). The reaction flask is refilled with nitrogen again and 500 g (3.5 mol) of phosphorus pentoxide is quickly added through an offset glass funnel while chlorofluorocarbon oil is stirred vigorously. The reaction flask is evacuated and refilled again. A 250 mL addition funnel is attached to the reaction flask and charged with 238 g (1.3 mol) of TFESA. TFESA is then added to the reaction mixture with good stirring over a period of 15 minutes. The flask becomes warm to the touch. The reaction is stirred at room temperature for 30 minutes. Internal pressure is lowered to 2.7 torr (360 Pa), the receiving flask of the distillation apparatus is chilled with liquid nitrogen and, after 15 minutes, several milliliters of clear colorless liquid are collected as a foreshot. A new receiver is placed in the apparatus and the pressure is lowered to 2 torr (266 Pa). A heating mantle is used to slowly apply heat to the flask. The temperature of the distillation is gradually increased from 50° C. to 130° C. One fraction, over a three hour period, is collected to give 167 g (73.8% yield) of 2-hydrotetrafluoroethanesulfonic acid anhydride. NMR analysis confirms the identity of the product.

### Example 3

### Preparation of 2-Hydrotetrafluoroethanesulfonic Acid Anhydride in Perfluoropolvether (PFPE) Oil

Example 2 is repeated with the substitution of Krytox® TLF 8996 oil for Halovac® 100. Krytox® TLF 8996 is a low viscosity perfluoropolyether of the general structure F[CF(CF₃)CF₂O]ₙCF₂CF₃ where n is about 5 to 11. 2-Hydrotetrafluoroethanesulfonic acid anhydride is obtained in a yield of 55%, less than the 73.8% yield in Example 2. This shows the superiority of the chlorofluorocarbon oil over PFPE oil in the preparation of 2-hydrotetrafluoroethanesulfonic acid anhydride from TFESA by reaction with P₂O₅.

### Example 4

### Preparation of Butyl-2-hydrotetrafluoroethanesulfonate

An oven-dried three-neck, 100 mL round-bottom flask, under nitrogen atmosphere, is charged with 30 mL of anhydrous dichloromethane and 0.53 mL (6.5 mmol) of anhydrous pyridine. The reaction mixture is cooled with an ethylene glycol/CO₂ (dry ice) bath to −30° C. A solution of 2.25 g (6.5 mmol) 2-hydrotetrafluoroethanesulfonic acid anhydride in 10 mL of anhydrous dichloromethane is prepared in a drybox and then added by syringe to the reaction mixture, keeping the temperature at −20° C. A solution of 0.28 g (3.8 mmol) of n-butanol in 5 mL of anhydrous dichloromethane is added to the cold reaction mixture. The temperature is maintained at −20° C. during the addition. The reaction is stirred cold for 75 min. Low-boiling volatiles are removed under reduced pressure. The desired product is distilled out of the residue to give 1.3 g (84%) of butyl 2-hydrotetrafluoroethane sulfonate as a clear colorless liquid. The identity of the ester is confirmed by proton and fluorine NMR. This example demonstrates the production in good yield of the butyl ester by reaction of butyl alcohol with 2-hydrotetrafluoroethanesulfonic acid anhydride. This is in contrast to the isomeric 1-hydrotetrafluoroethanesulfonic acid anhydride with which it is possible to make only the methyl and ethyl esters, higher alcohols giving olefin.

### Example 5

### Preparation of 4-tert-Butylphenol-2-hydrotetrafluoroethanesulfonate

An oven-dried three-neck, 250 mL round-bottom flask, under nitrogen atmosphere, is charged with 100 mL of anhydrous dichloromethane and 0.53 mL (6.5 mmol) of anhydrous pyridine. The reaction mixture is cooled with an ethylene glycol/CO₂ bath to −30° C. A solution of 2.25 g (6.5 mmol) 2-hydrotetrafluoroethanesulfonic acid anhydride in 20 mL of anhydrous dichloromethane is prepared in a drybox and then added by syringe to the reaction mixture keeping temperature at −20° C. A solution of 0.57 g (3.8 mmol) of 4-tert-butylphenol in 40 mL of anhydrous dichloromethane is added to the cold reaction mixture. The temperature is maintained at −20° C. during the addition. Temperature is maintained and stirring is continued for 75 min. By thin layer chromatography (TLC), using 25% ethyl acetate/hexane, 4-tert-butylphenol is found to be absent. Stirring is continued at −10° C. for a further 3 hours, then the reaction mixture is poured onto 200 mL of 5% NaHCO₃. The layers are separated and the organic layer is dried over Na₂SO₄. The solvent is removed and the crude product is purified on a silica gel column (10% ethyl acetate/hexane) to yield 1.0 g (84%) of 4-(tert-butylphenyl)-2-hydrotetrafluoroethane sulfonate as a clear colorless liquid.

NMR Analysis: ¹H NMR (CDCl₃) 1.32 (9H, s); 6.23 (1H, tt, ²J_{HF}=52.3 Hz); 7.19 (2H, app d); 7.43 (2H, app d). ¹⁹F NMR (CDCl₃) −117.15 (2F,m); −135.3 (2F, dt, ²J_{HF}=52.3 Hz).

Elementary Analysis: Calculated: % C 45.86; % H 4.49; % F, 24.18; % S, 10.2. Found: % C 46.24; % H 5.03; % F, 24.44; % S, 9.78.

### Example 6

### Preparation of N,N-dimethyl-2-hydrotetrafluoroethanesulfonamide

Following the general procedure of Example 4, 2-hydrotetrafluoroethanesulfonic acid anhydride is reacted with dimethyl amine in place of n-butanol. The product is substantially all N,N-dimethyl-2-hydrotetrafluoroethanesulfonamide with no detectable amounts of the product of reaction of 2-hydrotetrafluoroethanesulfonic acid anhydride with two molecules of dimethyl amine. This example demonstrates that 2-hydrotetrafluoroethanesulfonic acid anhydride reacts cleanly with amines to make amides without significant side reaction, unlike the isomeric 1-hydrotetrafluoroethanesulfonic acid anhydride in which reaction with two molecules of amine can be the predominant reaction.

### Example 7

### Preparation of N-(4-tert-butylphenyl)aniline

In a drybox, a glass thick-walled pressure tube is charged with 0.055 mL (0.6 mmoL) of aniline, 0.157 g (0.5 mmol) of 4-tert-butylphenyl 2-hydrotetrafluoroethane sulfonate, 0.067 g (0.7 mmol) of sodium t-butoxide, 0.02 g (0.05 mmol) of 2-(dicyclohexylphosphino)-2′-(N,N-dimethylamino)biphenyl, 0.023 g (0.025 mmol) of palladium dibenzylideneacetone and 2 mL of toluene. The tube is sealed, brought out of the drybox and heated at 80° C. for 16 h. The reaction mixture is cooled to room temperature, the tube is opened, and ether is added and the mixture is passed through a small plug of celite. The solvents are removed in vacuo. The crude product is purified on a 20 g silica gel column using 5% ethyl acetate/hexane. Removal of volatiles yields 0.096 g (86%) of N-(4-tert-butylphenyl)aniline as a light brown oil. The proton NMR is identical to that of N-(4-tert-butylphenyl)aniline as given in the literature. This reaction goes in good yield is in contrast to the reaction of 4-tert-butylphenyl 1-hydrotetrafluoroethane sulfonate with aniline, which gives a complex mixture of products.

What is claimed is:

1. Alkyl esters of the formula
R^{f}CH FCF₂SO₂OR⁴
where R^{f} is selected from the group consisting of Cl, F, a C₁ to C₄ perfluoroalkyl group, or a C₁ to C₄ perfluoroalkoxy group, and where R⁴ is a C₁ to C₁₂ linear, branched, or cyclic alkyl group or an C₇ to C₂₀ aryl-substituted alkyl group, and where said aryl group may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H and R².

2. The alkyl ester of claim 1 selected from the group consisting of CHF₂CF₂SO₂O-n-C₄H₉, CHClFCF₂SO₂OCH₂CH₂Cl, CF₃CHFCF₂SO₂OCH₂CH₂C₆H₅, CF₃OCHFCF₂SO₂O-2-C₃H₇, C₂F₅OCHFCF₂SO₂OCH₃, and C₂F₅CF₂OCHFCF₂SO₂OCH₂CH₂C₆F₁₃.

3. Aryl esters of the formula
R^{f}CH FCF₂SO₂OR¹
where R^{f} is selected from the group consisting of Cl, F, a C₁ to C₄ perfluoroalkyl group, or a C₁ to C₄ perfluoroalkoxy group, and where R¹ is a C₆ to C₁₄ aryl group, and where said aryl group may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H and R².

4. The aryl esters of claim 3 selected from the group consisting of CHF₂CF₂SO₂OC₆H₄-4-tert-C₄H₉, CHClFCF₂SO₂OC₆H₄-4-OCH₃, CF₃CH FCF₂SO₂OC₆H₄CF₃CH FCF₂SO₂OC₆H₄-3-CF₃, CF₃OCHFCF₂SO₂O-2-C₁₀H₇, C₂F₅OCHFCF₂SO₂OC₆H₄-4-CN, and C₂F₅CF₂OCHFCF₂SO₂OC₆H₅.

5. A process for preparing alkyl esters of the formula R^{f}CHFCF₂SO₂OR⁴ where R^{f} is selected from the group consisting of Cl, F, a C₁ to C₄ perfluoroalkyl group, or a C₁ to C₄ perfluoroalkoxy group, and where R⁴ is a C₁ to C₁₂ linear, branched, or cyclic alkyl group or an C₇ to C₂₀ aryl-substituted alkyl group, where the aryl group may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H and R², comprising contacting alcohol of the formula HOR⁴ with fluorinated alkanesulfonic acid anhydride of the formula (R^{f}CHFCF₂SO₂)₂O.

6. A process for preparing aryl esters of the formula R^{f}CHFCF₂SO₂OR¹ where R^{f} is selected from the group consisting of Cl, F, a C₁ to C₄ perfluoroalkyl group, or a C₁ to C₄ perfluoroalkoxy group, and where R¹ is a C₆ to C₁₄ aryl group, and where the aryl group may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H (hydrogen) and R², comprising contacting aromatic alcohol of the formula HOR¹ with fluorinated alkanesulfonic acid anhydride of the formula (R^{f}CHFCF₂SO₂)₂O.

7. Sulfonamides of the formula
R^{f}CHFCF₂SO₂N R⁵R⁶
where R^{f} is selected from the group consisting of Cl, F, a C₁ to C₄ perfluoroalkyl group, or a C₁ to C₄ perfluoroalkoxy group, and where R⁵ is a C₁ to C₁₂ linear, branched, or cyclic alkyl group, or a C₇ to C₂₀ aryl-substituted alkyl group, or a C₆ to C₁₄ aryl group, said aryl groups may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H and R², and where R⁶ is independently selected from the group consisting of H (hydrogen) and R⁵, and where R⁵ and R⁶ together may form a cyclic structure.

8. The sulfonamide of claim 7 selected from the group consisting of CHF₂CF₂SO₂N(CH₃)₂, CHClFCF₂SO₂NH(tert-C₄H₉), CF₃CHFCF₂SO₂NHC₆H₅, CF₃OCHFCF₂SO₂N(C₂H₅)₂, C₂F₅OCHFCF₂SO₂NH(C₆H₄-3-CF₃), and C₂F₅CF₂OCHFCF₂SO₂NHC₆H₄-4-C₆H₅.

9. A process for preparing sulfonamides of the formula
R^{f}CH FCF₂SO₂N R⁵R⁶
where R^{f} is selected from the group consisting of Cl, F, a C₁ to C₄ perfluoroalkyl group, or a C₁ to C₄ perfluoroalkoxy group, and where R⁵ is a C₁ to C₁₂ linear, branched, or cyclic alkyl group, or a C₇ to C₂₀ aryl-substituted alkyl group, or a C₆ to C₁₄ aryl group, said aryl groups may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H and R², and where R⁶ is independently selected from the group consisting of H (hydrogen) and R⁵, and where R⁵ and R⁶ together may form a cyclic structure, comprising contacting amine of the formula NHR⁵R⁶ with a fluorinated alkanesulfonic acid anhydride of the formula (R^{f}CHFCF₂SO₂)₂O.

10. A process for the arylation of an amine comprising contacting an amine of the formula NHR⁵R⁶ with an aryl ester of the formula R^{f}CHFCF₂SO₂OR¹ in the presence of a palladium catalyst to form an amine of the formula NR¹R⁵R⁶, where R^{f} is selected from the group consisting of Cl, F, a C₁ to C₄ perfluoroalkyl group, or a C₁ to C₄ perfluoroalkoxy group, and where R⁵ is a C₁ to C₁₂ linear, branched, or cyclic alkyl group, or a C₇ to C₂₀ aryl-substituted alkyl group, or a C₆ to C₁₄ aryl group, said aryl groups may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H and R², and where R⁶ is independently selected from the group consisting of H and R⁵, and where R⁵ and R⁶ together may form a cyclic structure and R¹ is a C₆ to C₁₄ aryl group, and where said aryl group may be further substituted with one or more substituents selected from the group consisting of R², F, Cl, Br, I, NO₂, CN, OR, C(O)R , CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H and R².

11. A process for the alkylation of an amine comprising contacting an amine of the formula NHR⁵R⁶ with an alkyl ester of the formula R^{f}CHFCF₂SO₂OR⁴ to form an amine of the formula NR⁴R⁵R⁶, where R^{f} is selected from the group consisting of Cl, F, a C₁ to C₄ perfluoroalkyl group, or a C₁ to C₄ perfluoroalkoxy group, and where R⁴is a C₁ to C₁₂ linear, branched, or cyclic alkyl group or an C₇ to C₂₀ aryl-substituted alkyl group, and where said aryl groups may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H (hydrogen) and R², and where R⁵ is a C₁ to C₁₂ linear, branched, or cyclic alkyl group, or a C₇ to C₂₀ aryl-substituted alkyl group, or a C₆ to C₁₄ aryl group, said aryl groups may be further substituted with one or more substituents selected from the group consisting of R², F, Cl, Br, I, NO₂, CN, OR², C(O)R², CO₂R², C(O)NR²R³, NR²R³, NHC(O)R², NHC(O)NR²R³, SO₂R², or SO₂NR²R³, where R² is a C₁ to C₁₂ linear, branched, or cyclic alkyl group, a C₆ to C₁₄ aryl group, a C₇ to C₂₀ alkyl-substituted aryl group, and C₇ to C₂₀ aryl-substituted alkyl group, and R³ is independently selected from the group consisting of H (hydrogen) and R², and where R⁶ is independently selected from the group consisting of H and R⁵, and where R⁵ and R⁶ together may form a cyclic structure.
